(19) United States
(12) Patent Application Publication
(10) Pub. No.: US 2008/0058637 A1
(43) Pub. Date: Mar. 6, 2008

(54) Means and method for marking tissue in a human subject

(76) Inventors: Robert E. Fischell, Dayton, MD (US); David J. Mullen, Old Greenwich, CT (US)

Correspondence Address: Dr. Robert E. Fischell, 14600 Viburnum Drive, Dayton, MD, 21036, US

(21) Appl. No.: 11/502,960

(22) Filed: Aug. 14, 2006

(51) Int. Cl. A61B 5/05

(52) U.S. Cl. 600/424

(57) ABSTRACT

Disclosed is a system for placing an elongated permanent magnet into a volume of tissue within a human body that is suspected of being cancerous. The magnet's position is then detected by a magnet locating system that is used by a surgeon to find and remove the suspected tissue volume including a margin of tissue to assure that any and all cancer cells have been excised. The magnet locating system includes a wireless probe that sends a signal to a range indication device that indicates the range from the end of the probe to the magnet implanted in the human body. This indication of distance to the magnet is preferably accomplished by an audio signal or a visual display.

### FIELD OF USE

This invention is in the field of methods and devices for marking human tissue that may be cancerous so that tissue can be excised by a surgeon.

### BACKGROUND OF THE INVENTION

The need for accurate preoperative image guided localization of nonpalpable breast lesions has been well described, and the frequency of use for this technique is increasing. Not only mammographically detected lesions require localization, but also lesions that may be found by any other imaging technique such as ultrasound, MRI, nuclear medicine or other technologies not yet described. Such localizations generally require the positioning of a temporary marker, most frequently constructed of a metal anchor on the end of a wire inserted through a needle that has been accurately positioned by image guidance prior to the release of the marker. See, Frank H. A., Hall F. M., Steer M. L., Preoperative Localization of Nonpalpable Breast Lesions Demonstrated by Mammography; New England Journal of Medicine, 1976; 296:259-260. In addition, the implantation of a small metal “clip” marker following large core biopsy under image guidance may be used when the visualized target has been substantially removed during the diagnostic procedure (thus compromising future successful localization). See, Burbank F. et al., “Tissue Marking Clip for Stereotactic Breast Biopsy: Initial Placement Accuracy, Long-term Stability, and Usefulness as a Guide for Wire Localization”; Radiology, 1997; 205:407-415.

The need for such localization is best understood in the breast but will be of growing importance in other organ systems particularly for marking suspected tissue volume in the lungs. The explosive growth of diagnostic imaging has increased the frequency of detection of small lesions throughout the body that cannot be seen or felt by the surgeon who is charged with the task of removing the suspected tissue volume. Guidance for radiation therapy or other emerging ablation techniques using thermal, laser, radiofrequency or other methods of local energy or drug deposition to kill cells is also needed.

Many metal devices to accomplish breast marking or localization have been devised (e.g., U.S. Pat. Nos. 4,799,495; 5,011,473; 5,057,085; 5,083,570; 5,127,916; 5,158,084; 5,221,269; 5,234,426; 5,409,004; 5,556,410; 6,053,925; and 6,544,269). These devices all have the significant limitation of requiring the image guided localization procedure immediately before the surgery. Because the anchoring device is connected to a wire that protrudes through the skin, it must be promptly removed. Even those devices now approved for implantation into the breast (following the special case of a small lesion which has been substantially removed during image guided large core needle biopsy) must be re-localized with a second temporary device on the day of definitive surgery. The need for immediate preoperative localization creates logistical problems for radiology departments and operating room personnel. Any system that could eliminate the need for prompt surgery after localization of the suspected tissue volume would be an advance in this field.

Thus, a device that could be implanted by a radiologist at one time and then independently removed by a surgeon at another time on the day of the needed surgical procedure with no further patient preparation is desirable. Such a device should serve as a marker placed when the need for surgical guidance is already specifically known. Additionally, such a device should be able to mark the site of a large core percutaneous biopsy when the need for surgery at some future point is considered likely. Although some have attempted solutions to these problems, a simple and cost effective approach has not yet been found.

Before the era of diagnostic breast imaging, only palpable lesions were detectable. Palpable lesions can be biopsied by a surgeon without any form of marking or guidance other than physical examination of the breast before and during the surgical procedure. Lesions that are detectable only by imaging, however, are best biopsied after marking. Although this is now done following wire localization, some have suggested the use of markers that may render a previously nonpalpable lesion palpable, thus providing the surgeon with a familiar method of tactile guidance. See, Debbas, Apparatus for Locating a Breast Mass, U.S. Pat. No. 5,662,674; Fulton et al. Biopsy Localization Method and Device, U.S. Pat. No. 6,730,042; and Fulton et al. Target Tissue Localization Device and Method, U.S. Pat. No. 6,409,742. These proposed devices all have one significant drawback in common: they are large and may be expected to be uncomfortable for patients. This problem may be further compounded when these devices must remain in position for some length of time.

In PCT Patent Application No. PCT/US04/37605, D. J. Mullen describes a means for the surgeon to find a suspected tissue volume using a metal detector that detects wires that have been implanted within the suspected tissue volume. Although this system is workable, the metal detector for such small wires can only detect them within a comparatively close range of the suspected tissue volume.

In U.S. Pat. No. 6,698,433, D. N. Krag describes several means to localize a suspected tissue volume. These methods include the placement of a multiplicity of tiny magnets around the suspected tissue volume. Specifically, in FIG. 1 of the Krag patent, six small objects that could be permanent magnets are located around the suspected tissue volume. In FIG. 1A, ten small objects that could be permanent magnets are placed around a suspected tissue volume. In FIG. 2b of the Krag patent he shows a tiny magnet which has a diameter that is actually greater in its transverse dimension as compared to the length of the magnet between the magnetic poles. That is, the ratio of the distance between the poles compared to the magnet diameter is less than 1.0. Such tiny magnets have an extremely small field at any reasonable distance outside of a human breast because the close proximity of the north and south poles result in cancellation of the magnet's external field. A collection of six to ten such magnets randomly placed around a suspected tissue volume would not be at all detectable using a magnetometer probe outside of the breast. This is in contradistinction to a single (or even two or three) elongated magnets that have a magnetic field that would be readily detectable outside of the breast. What is required but not at all anticipated by the teachings of the Krag patent is an elongated magnet that has a distance between the poles that is at least three times the magnet's diameter and preferably at least 5 to 10 times the magnet's diameter.

### SUMMARY OF THE INVENTION

Disclosed herein is a system for placing an elongated permanent magnet into a volume of tissue within a human body that is suspected of being cancerous. The magnet's position is then detected by a magnet locating system that is used by a surgeon to find and remove the suspected tissue volume including a margin of tissue to assure that any and all cancer cells have been excised.

A first method to accomplish the placement of the magnet is to use a magnet injector that has a sharpened cannula with the magnet placed at the cannula's distal end. One of several different methods can be used by a radiologist to determine the location of the suspected tissue volume. These methods include sonography, fluoroscopy, MRI and mammography. After the injector places the magnet within the suspected tissue volume, a spring release mechanism is pulled out of the handle of the injector and a spring within the handle quickly removes the cannula from the breast. This leaves the magnet in place for detection by a magnetometer probe that is operated by a surgeon. A preferred embodiment of the system is that the magnet extends beyond the extremities of the suspected tissue volume by a length that is approximately equal to the tissue margin that the surgeon hopes to attain. Thus when the magnet is removed, the suspected tissue volume plus the additional margin tissue is also removed.

Locating the magnet within the breast is accomplished by means of a magnetometer probe that can take several different forms. One form is a total field magnetometer located near the distal end of the probe. Another embodiment of the magnet locating system is to use a vector magnetometer or a vector magnetic gradient detector. Such a gradient detector has the advantage of being able to automatically cancel out the earth's magnetic field and any local field whose gradient is (as would be expected) much smaller than the gradient of the magnetic field from the implanted magnet.

To assist the surgeon in detecting the location of the magnet, it is desirable to place the magnet so as to be parallel to the patient's chest wall (i.e., horizontal) with the patient lying down and with the orientation of the long axis of the magnet being known to the surgeon, for example with the long axis of the magnet being perpendicular to the body's long axis. This is particularly valuable when a vector magnetic gradient magnetometer is used to detect the position of the magnet within the breast.

A second method for placing the magnet within the suspected tissue volume is to first use a sharp stylet within a cannula needle to penetrate through the suspected tissue volume. The stylet is then removed from the cannula needle that is placed through the suspected tissue volume. A magnet injector with a squared off distal end of its cannula and having the magnet at the injector cannula's distal end is then placed within the cannula needle that is already placed through the suspected tissue volume. The cannula needle is then pulled out of the breast. The elongated magnet within the cannula of the injector is then centered within the suspected tissue volume. The spring release handle is then pulled out of the handle of the magnet injector and a spring within the handle of the magnet injector pulls the cannula out of the breast leaving the magnet in place through the suspected tissue volume.

Thus one object of the present invention is to use a single, elongated permanent magnet to mark the location of a suspected tissue volume that may be cancerous.

Another object of this invention is to use a magnetometer probe to detect the position of the magnet within the suspected tissue volume.

Still another object of this invention is to use a vector magnetic gradient magnetometer to detect the position of the magnet so as to negate the effect of the earth's magnetic field and any local magnetic field.

Still another object of this invention is to use an elongated magnet that extends beyond the boundaries of the suspected tissue volume by a length approximately equal to the margin of normal tissue that should be removed around the suspected tissue volume to assure that no malignant cells remain in the body.

Still another object of this invention is to use a wireless probe system so that the probe can be operated by the surgeon without the encumbrance of a wire that connects the probe to the visual or audio means that indicates to the surgeon that he/she is approaching the implanted magnet.

Still another object of this invention is to use the systems and methods described herein to place a magnet into any suspected tissue volume within a human body that is to be excised by a surgeon.

These and other objects and advantages of this invention will become obvious to a person of ordinary skill in this art upon reading the detailed description of this invention including the associated drawings as presented herein.

### BRIEF DESCRIPTION OF THE DRAWINGS

FIG. 1 is a longitudinal cross section of a magnet injector that is used to place an elongated magnet within human tissue that may be cancerous.

FIG. 2 is a plan view of an anchor that is attached to the end of the magnet.

FIG. 3 is a transverse cross section of the magnetic injector handle at section 3-3 of FIG. 1 showing the means to release the spring that ejects the cannula from the breast.

FIG. 4 illustrates the magnetic field of a magnet placed into a female breast at the site of a suspected tissue volume. FIG. 4 also shows a magnet locating system for detecting the position of the magnet within the breast.

FIG. 5 shows a stylet and cannula needle system that is placed through a suspected tissue volume.

FIG. 6 shows a magnet injector placed within the cannula that was placed through the breast after the stylet was removed.

FIG. 7 shows the cannula of the insertion needle having been removed from the breast and slid along the cannula of the magnet injector.

FIG. 8 shows the magnet centered around the suspected tissue volume within the breast with the magnet anchors deployed.

FIG. 9 illustrates a permanent magnet located near the center of the type of wire that is currently used to mark a suspected tissue volume within the breast.

### DETAILED DESCRIPTION OF THE INVENTION

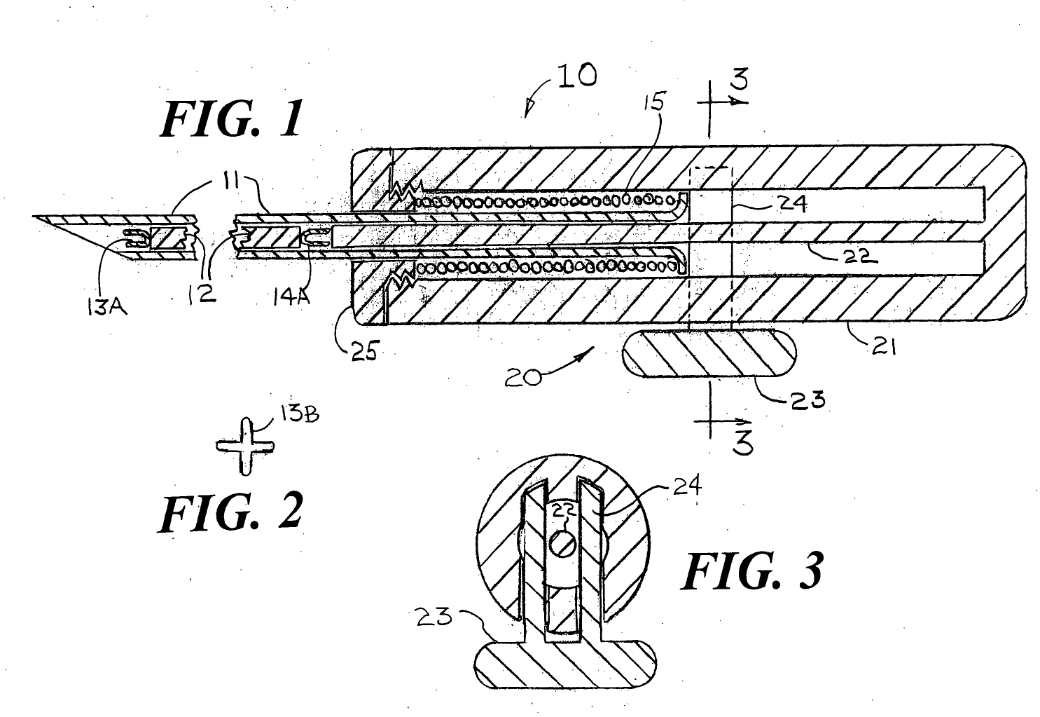

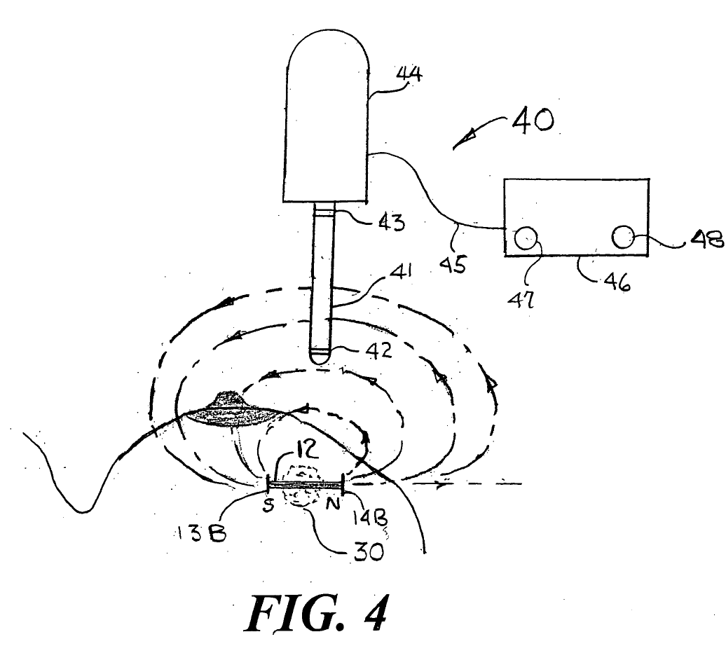

FIG. 1 shows a magnet injector 10 having a cannula 11 into which is placed a permanent magnet 12 having a pre-deployed distal anchor 13A fixedly attached at the magnet's distal end and a pre-deployed proximal anchor 14A fixedly attached at the magnet's proximal end. It is also conceived that the magnet could have one or more anchors deployed near the center of the magnet that also would be used to stabilize the position of the magnet within the breast. The shape of the deployed distal anchor 13B is shown in FIG. 2. The cannula 11 is positioned within a handle 20 that has an outer cylinder 21, an inner cylinder 22 and a spring release handle 23 that is attached to a spring holder 24 that keeps the spring 15 from expanding until the magnet 12 is ready for release within the suspected tissue volume. The distal end of the spring 15 is held by the end cap 25 and the proximal end of the spring is held by an expanded end section of the cannula 11. When the magnet 12 is placed into a suspected tissue volume 30 (see FIG. 4), the spring release handle 23 is pulled out of the outer cylinder 21 which removes the spring holder 24 which causes the cannula 11 to be pulled into the handle 20 while the inner cylinder 22 causes the magnet 12 to be accurately retained within the suspected tissue volume 30 as shown in FIG. 4.

The cannula 11 would typically be made from thin-walled stainless steel with an inside diameter that typically would be just slightly larger than 1.0 mm. A range of diameters for the cannula 11 from 0.3 to 3.0 mm is certainly possible for this application. The magnet 12 would be made from any permanent magnetic material having a reasonably high energy product. A preferred magnetic material would be strong and ductile. A preferred permanent magnet material would be the alloy “Vicalloy.”

FIG. 4 shows a human breast into which the magnet 12 has been placed into the suspected tissue volume 30 by the injector 10 of FIG. 1 or the injector 60 of FIG. 6. FIG. 4 also shows the deployed anchors 13B and 14B. The anchors 13B and 14B are optimally formed from a shape memory alloy such as Nitinol. Optimally, the Nitinol would have a transition temperature of about 90 degrees F. so that it could be welded or otherwise fixedly attached to the ends of the magnet 12 and then bent so as to be easily placed within the cannula 11. After the predeployed anchors 13A and 14A of FIG. 1 are placed into breast tissue, the temperature of the breast will cause the anchors to deploy to the shape that is shown in FIGS. 2 and 4. The function of these anchors is to prevent shifting of the magnet within the breast after it has been accurately placed within the suspected tissue volume 30. In order to get a reasonably high level of magnetic field outside of the breast, it is necessary for the magnet 12 to have a reasonably high energy product and also that the magnet 12 have a reasonably long length for the distance between the magnetic north pole, N and the south magnetic pole, S. This ratio of length between the poles to the diameter of the magnet 12 should be at least 3:1 and preferably in the range of at least 5:1. It is ideal for the magnet 12 to protrude beyond the suspected tissue volume 30 by at least 2 mm so that there will be a margin of normal tissue between the perimeter of the suspected tissue volume and the end of the magnet 12. An optimal size for the magnet 12 would have it extend for approximately 5 mm beyond the outer surface of the suspected tissue volume 30. Therefore, the magnet 12 should be at least 10 mm long to provide an optimum margin for the very smallest suspected tissue volume 30. The surgeon who would be removing the magnet 12 and the suspected tissue volume 30 would be guided by the extremities of the magnet 12 for the removal of tissue with an adequate margin. Specifically, the surgeon should remove a volume of tissue that has a spherical diameter that is approximately equal to the distance between the deployed anchors 13B and 14B. If the suspected tissue volume 30 is not at all spherical in its extent, the surgeon could be advised of that shape so that the appropriate tissue excision could be accomplished. It is also conceived that 2 or 3 elongated magnets could be used to define a suspected tissue volume that is not small and is of an odd shape. However, the typical suspected tissue with modern mammography is sufficiently small so that a spherical excision of tissue to the diameter which is essentially the length of the magnet 12 will be generally sufficient to maintain an adequate tissue margin.

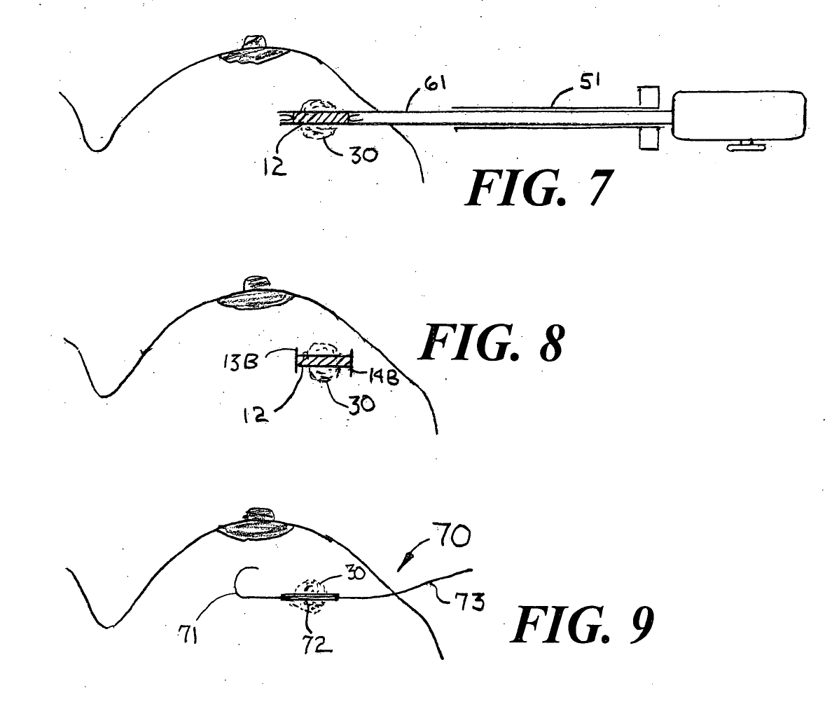

The use of the deployed anchors 13B and 14B of FIG. 4 represents a unique aspect of the invention to provide centering of the magnet 12 with respect to the tissue volume 30 under varied deployment conditions. Needle localization is generally accomplished using mammographic guidance. The use of stereotactic needle localization has been described, but carries with it certain significant limitations. These limitations are predominantly due to the fact that, although accurate localization within millimeters of the target is readily accomplished while the breast is compressed within the stereotactic apparatus, upon release of compression significant errors in the depth of marker placement are frequently encountered. This limitation has been understood as the so called “accordion” effect and is due to the re-expansion of the breast after the release of compression and often results in difficulty maintaining precise localization of a marker after the release of compression. In one embodiment of the present invention this limitation is overcome, since the presence of deployed anchors 13B and 14B (of FIGS. 4 and 8) at the distal as well as the proximal ends of the magnet 12 will prevent both forward and reverse migration of the magnet 12 with respect to the tissue volume 30. By this means, accurate localization will be reliably maintained after the breast is released from compression. Thus, an advantage of a device according to the present invention is that it will allow accurate tissue localization to be easily accomplished using stereotactic mammography equipment, significantly increasing the range and number of procedures that can be performed on stereotactic mammography equipment. The stereotactic mammography equipment itself, as well as the rooms in which this equipment is located, represents a significant capital expense for a facility offering such procedures. Allowing localizations to be performed on such equipment enhances the productivity of these rooms. Furthermore, patients will experience significant benefits in the use of this embodiment of the inventive procedure. It is well known that the prone position offered by stereotactic mammography improves patients' tolerance of needle procedures in the breast by decreasing the incidence of vasovagal responses as well as the perception of pain. Procedure time is significantly decreased by the use of digital receptors and precision is improved by the stereotactic technique.

FIG. 4 also shows a magnet locating system 40 that includes a magnetometer probe 41, an end magnetic field detector 42, a reference magnetic field detector 43, and a handle 44 that is connected by a wire 45 to the electronic module and range indicator 46. The end magnetic field detector 42 can be either a total field magnetometer or a vector magnetometer that would measure only one component of the magnetic field from the implanted magnet 12. If the end magnetic field detector 42 is a total field magnetometer, then the magnet locating system 40 would have a means to null out the earth's field and any local magnetic field where the patient is situated. If a single axis, vector magnetometer is used, then the reference detector 43 must also be a vector magnetometer that measures the magnetic field intensity in the same direction as the end magnetic field detector 42. The preferred embodiment of the magnet locating system 40 would utilize the detectors 42 and 43 as a vector magnetic gradiometer which measures the difference in the vector magnetic field intensity at the position of the detector 42 compared to the vector magnetic field intensity at the position of the detector 43. In this way both the earth's field is automatically cancelled as well as any local field that has a gradient that would typically be much lower as compared to the gradient of the magnetic field intensity from the magnet 12. The end magnetic field detector 42 and the reference detector 43 should be able to detect a magnetic field intensity as low as 0.1 milligauss. The separation between the detectors 42 and 43 should be at least 2 cm and optimally between 5 and 10 cm. The magnetometer probe 41 could use any one of a variety of magnetic field detectors which could be Hall effect, fluxgate or giant magneto resistance (GMR) sensors. Of these, the GMR sensor is preferred because of its small size.

The electronics module and range indicator 46 would receive a signal from the magnetometer probe 41 that is indicative of the magnetic field intensity at the position of the end magnetic field detector 42. The output of the range indicator 46 could be an audio signal or a visual display or both. The audio signal could be an audio tone that was (for example) a higher pitched sound as the magnetic field detector 42 approached the magnet 12. Another audio detection means would be a succession of pulses that had a shorter time between pulses as the detector 42 gets closer to the magnet. When the detector is within a short distance from the magnet 12, the sound could become continuous. Once the tone was continuous, it is conceivable that, at that point, the pitch of the sound would become a higher frequency as the magnet 12 was approached. If a visual display was used to tell the surgeon that he/she was approaching the magnet 12, it could be (for example) a meter that gives the approximate distance in millimeters or centimeters between the end of the probe 41 and the center of the magnet 12.

FIG. 4 also shows that the electronics module and range indicator 46 has a magnetic length adjustment knob 47 and a magnetic dipole moment adjustment knob 48. Each magnet 12 that is placed within a breast will have on its package a listing of the magnet's length and its magnetic dipole moment. In order to accurately determine the distance to the magnet 12, the range indicator 46 will have an adjustment for these two aspects of the magnet 12 that will correlate the range to that magnet 12 and the position of the probe 41. The knobs 47 and 48 will provide that adjustment.

A preferred embodiment of the magnet locating system 40 would use a wireless connection between the probe 41 and the range indicator 46, i.e., there would not be a wire 45 connecting the probe 41 to the range indicator 46. In this way, the surgeon could grasp the probe handle 44 and move it to seek the magnet 12 without being constrained in any way by the connecting wire 45. A wireless connection would allow the range indicator 46 to be placed at any convenient place in the operating room without being constrained by a connection to the wire 45. Another preferred embodiment of this invention would have all the electronics and source of audio or visual indication of range to the magnet 12 as part of the probe 41. Thus there would be no requirement for either a wire 45 or a radio transmitter in the probe 41 with a radio receiver in the range indicator 46. The magnet locating system 40 could be operated by either rechargeable or primary batteries. Not shown in FIG. 4 but required for the magnet locating system 40 are ON-OFF switches that turn the magnetometer probe 41 and the range indicator 46 either on or off. Also, a LOW BATTERY LIGHT is envisioned to tell the operating room personnel that new batteries are required. Still further, it is envisioned to have a volume control on the range indicator 46 to optimize the sound level desired by the surgeon during the procedure to excise the suspected tissue volume 30.

A removable sterile plastic cover could be placed over the probe 41 including the handle 44 before the surgeon utilizes that magnetic field probe. This would better assure sterile conditions for the excision of the magnet 12 and the suspected tissue volume 30. The use of a wireless connection would make it easier to maintain sterility because there would be no connecting wire 45 that could compromise the required sterile conditions for this procedure. Although the magnet injector 10 is a disposable device that is sterilized by the manufacturer, the magnet locating system 40 is not disposable and therefore a means must be provided for at least the probe 41 that is handled by the surgeon to be made sterile for every surgical procedure. The optimum means for attaining sterility is for a sterile cover to be placed over the probe 41 before the procedure and the range indicator would not be covered but would not be touched by the surgeon during the procedure.

When the magnet 12 is placed into the breast, it would be optimum to have it placed parallel to the patient's chest wall and for the surgeon to know the magnet's orientation so the detector 42 may be rapidly aligned with the implant. A standardized method of insertion either from right to left for the patient's right breast or from left to right for the patient's left breast can provide an optimum method for placement of the magnet 12 by routinely placing the long axis of the magnet 12 parallel to the chest wall and perpendicular to a line from the patient's head to the patient's feet. Alternatively, other orientations of the implant parallel to the chest wall may be equally successfully utilized if the implant orientation is demonstrated by the alignment of the points of a magnetic compass with the poles of the magnet 12 that may be observed while moving the compass over the marked breast, marked on the patient's skin after implantation, or pictorially described by the radiologist and communicated to the surgeon. With the prior knowledge of the orientation of the magnet 12, the surgeon using the magnet locating system 40 would immediately be able to orient the probe 41 to best locate the magnet 12. For example, if the preferred embodiment of a vector magnetic gradiometer is used, the detector 42 could be positioned to detect a magnetic field component from the magnet 12 implanted parallel to the patient's chest wall by simply aiming the probe 41 perpendicular to the body's long axis with the detector 42 aligned with the magnet 12. Such prior knowledge of the position of the magnet 12 would speed up the detection of its position within the breast.

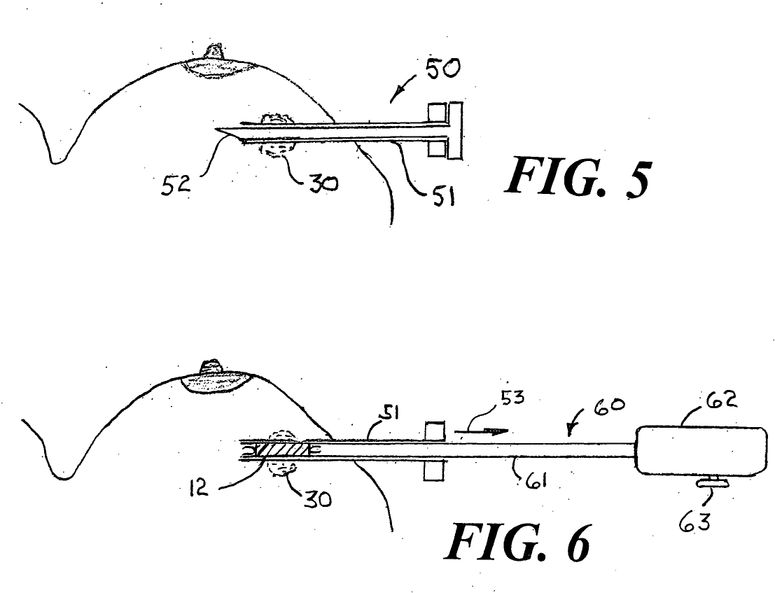

FIGS. 5-8 show an alternative method for placing the magnet 12 into the optimum position in a woman's breast. FIG. 5 shows a needle system 50 consisting of a needle cannula 51 with a square end into which a pointed stylet 52 has been placed. Although FIG. 5 shows a stylet with an angled end point, a stylet 52 with a diamond shaped point can be at least equally effective in placing the needle system into the breast. Because the needle system is smaller and lighter and easier to handle as compared to the magnet injector 10 of FIG. 1, it could be advantageously used in certain circumstances to penetrate through the suspected tissue volume 30. As shown in FIG. 6, the next stage in placing a magnet 12 into the correct position within the suspected tissue volume 30 would be to use a magnet injector 60 to place the magnet 12 at the distal end of the cannula 51. This would be done after the stylet 52 was removed from the cannula 51. The injector 60 differs from the injector 10 in that its distal end is square and not pointed as is the shape of the distal end of the injector 10. The magnet 12 within the cannula 61 is placed within the cannula 51 so as to be centered over the suspected tissue volume 30. Further, a needle system 50 may be made of MRI compatible materials (for example, titanium or non-magnetic stainless steel) thus allowing accurate positioning of the needle system 50 while the patient is within the strong magnetic field of an MRI machine and chamber. By first positioning a MRI compatible needle system 50 under MRI guidance, removing the patient from the strong magnetic field inside the MRI chamber and then using the magnet injector 60 outside of the MRI chamber, a magnet 12 may be accurately positioned without the need to ever bring the magnet 12 near the MRI magnetic field. In this fashion, the well known hazard of projectile movement of a magnet 12 brought near the strong magnetic field used in MRI will be avoided.

FIG. 7 shows the cannula 51 removed from the breast and slid onto the cannula 61 of the magnet injector 60. This is accomplished by holding the handle 62 of the injector 60 while pulling the cannula 51 out of the breast. The next step in this method for placing the magnet 12 through the suspected tissue volume 30 is to pull out the spring release handle 63 so that the spring (as shown in FIG. 1) pulls the cannula 61 out of the breast. The goal of having the magnet 12 with deployed anchors 13B and 14B is shown in FIG. 8. The magnet locating system 40 as shown in FIG. 4 can then be used by the surgeon to locate the magnet 12 within the woman's breast.

FIG. 9 illustrates an alternative embodiment of the present invention that uses a wire 70 with a hook 71 at its distal end and a proximal portion 73 that protrudes through the patient's skin. Except for the placement of a permanent magnet 72 at the center of the wire, this type of wire is what is typically used to mark a suspected tissue volume 30. The magnet 72 of FIG. 9 is shown without any tissue anchors that would be equivalent to the anchors 13B and 14B of FIG. 8. However, it should be understood that the magnet 72 could have either one or both anchors that are deployed like the anchors 13B and 14B of FIGS. 4 and 8. These anchors would prevent the axial migration of the wire 70 that does occasionally occur during localization. It should also be understood that the placement of such anchors would be a valuable improvement for the wire 70 even if the permanent magnet 72 was not used for localization. Although FIG. 9 shows that the wire 70 includes a hook 71, when anchors such as anchors 13B and 14B are used, the wire 70 could advantageously be straight and the hook 71 would not be needed to maintain accurate positioning of the magnet 72.

Any of the magnet placement systems described herein can be used to place the magnet 12 into certain suspected tissue volumes within a human body. For example, the systems described herein could be used by a radiologist to place a magnet 12 into a suspected tissue volume within a lung where a lesion is located that may be lung cancer. It is also envisioned to use the systems described herein at any location within a human body that is accessible by the injectors 10 or 60 for marking tissue that may be cancerous. Further, a magnet locating system 40 may be easily modified to allow for its placement on or through a thoracoscope, laparoscope or endoscope to allow the magnet locating system 40 to detect a magnet 12 placed in any location within the human body and thus aid in the successful removal of such marked tissues during minimally invasive thoracoscopic, laparoscopic or endoscopic procedures.

Although the magnet 12 described herein is optimally in the form of an elongated cylinder, the cross section of the magnet 12 could have any shape including square, hexagonal, octagonal, etc. For any such cross section, the largest transverse dimension can be considered to be the diameter of the elongated magnet 12.

Although a single magnet will be optimum for locating a typically small suspected tissue volume 30, it should be understood that two or more magnets could be used for localization of a large and/or complex suspected tissue volume 30. Furthermore, it should be understood that an optimum design for the magnet 12 or 72 would be to have it plated with a metal that is both highly radiopaque and compatible with human tissue. An optimum plating would be gold, platinum or tantalum all of which are human tissue compatible and highly radiopaque.

Various other modifications, adaptations and alternative designs are of course possible in light of the teachings as presented herein. Therefore it should be understood that, while still remaining within the scope and meaning of the appended claims, this invention could be practiced in a manner other than that which is specifically described herein.

What is claimed is:

1. A system for indicating the position of a suspected tissue volume within a human subject, the system including:
an elongated permanent magnet having a length of at least 10 mm and having a length between the magnet's north and south poles that is at least three times the magnet's diameter, the elongated permanent magnet being implanted within the suspected tissue volume within a human subject; and
a magnet locating system having a probe to detect the magnetic field from the permanent magnet, the magnet locating system also having electronic circuitry that creates either an audio signal or a visual display that indicates the distance from some point on the magnetic probe to the implanted magnet.

2. The system of claim 1 where the magnet is formed from Vicalloy.

3. The system of claim 1 where the magnet has an anchor fixedly attached to at least one of its ends.

4. The system of claim 1 where the magnet has at least one anchor placed near the longitudinal center of the magnet.

5. The system of claim 3 where the anchor is formed from a shape memory alloy having a transition temperature that is just below body temperature.

6. The system of claim 1 where the magnet is plated with a biocompatible metal that is also highly radiopaque.

7. The system of claim 6 where the plating on the magnet is gold.

8. The system of claim 1 where the suspected tissue volume is within the female breast.

9. The system of claim 1 where the suspected tissue volume is within a human lung.

10. The system of claim 1 where the probe of the magnet locating system is connected by a wire to the electronic circuitry that creates the audio signal or the visual display that indicates the distance of the probe from the implanted magnet.

11. The system of claim 1 where the probe of the magnet locating system has a wireless connection to the electronic circuitry that creates the audio signal or the visual display that indicates the distance of the probe from the implanted magnet.

12. The system of claim 1 where the probe contains all of the electronic systems including the range indication circuitry and audio or visual range indication means.

13. The system of claim 1 including a sterile cover that can be placed over the probe to assure the sterility of the surgery to remove the suspected tissue volume.

14. The system of claim 1 where the audio signal indicates the distance from the probe to the implanted magnet by the changing pitch of the audio signal.

15. The system of claim 1 where the audio signal indicates the distance from the probe to the implanted magnet by the time between sound pulses.

16. The system of claim 1 where the audio signal indicates the distance from the probe to the implanted magnet by the time between sound pulses and also a changing pitch of the sound as the probe closely approaches the magnet.

17. The system of claim 1 where the visual indication is a meter that indicates the distance from the probe to the implanted magnet.

18. The system of claim 1 further including a magnet injector that is designed to accurately place the magnet within the suspected tissue volume.

19. The system of claim 18 where the magnet injector has a handle into which a cannula is situated, the cannula having a distal end containing the elongated permanent magnet.

20. The system of claim 19 where inside the handle of the magnetic injector there is a spring and the handle also has a spring release mechanism to quickly withdraw the cannula from the suspected tissue volume.

21. The system of claim 1 where the magnet is placed inside the breast at the center of an elongated wire that has a proximal portion that extends outward through the skin of the human subject.

22. The system of claim 21 further including anchors fixedly attached to at least one of the ends of the permanent magnet.

23. The system of claim 1 where the probe of the magnet locating system is adapted for placement though an instrument that is selected from the group consisting of a thoracoscope, laparoscope and endoscope.

24. The system of claim 1 where the probe of the magnet locating system is adapted to be fixedly attached at or near the end of an instrument that is selected from the group consisting of a thoracoscope, laparoscope and endoscope.

25. A method for indicating the position of a suspected tissue volume within a human subject, the method including the following steps:
a) placing an elongated permanent magnet having a length of at least 10 mm within a cannula of a magnet injector;
b) using the magnetic injector to implant the elongated permanent magnet into the suspected tissue volume within the human subject;
c) using the magnetic injector to remove the cannula from the human subject while leaving the magnet in place within the suspected tissue volume;
d) detecting the magnetic field of the permanent magnet by means of a magnet locating system that has a probe that can detect a magnetic field and also has electronic circuitry that creates either an audio signal or a visual display that indicates the distance from some point on the magnetic field probe to the implanted magnet; and
e) surgically removing the magnet and the suspected tissue volume from the human subject.
